(19) United States
(12) Patent Application Publication
(10) Pub. No.: US 2026/0151133 A1
(43) Pub. Date: Jun. 4, 2026

(54) LIQUID METAL EMBOLIC FOR ANEURYSM TREATMENT WITH OPTIONAL LIQUID METAL EMBOLIC SEEPAGE PREVENTATIVE ELEMENT

(71) Applicant: DePuy Synthes Products, Inc., Raynham, MA (US)

(72) Inventors: Lacey GOROCHOW, Miami, FL (US); Daniel SOLAUN, Miami, FL (US); Pedro D. PEDROSO, Parkland, FL (US)

(73) Assignee: DePuy Synthes Products, Inc., Raynham, MA (US)

(21) Appl. No.: 18/898,805

(22) Filed: Sep. 27, 2024

(51) Int. Cl. A61B 17/12; A61L 24/02; A61L 24/06

(52) U.S. Cl. CPC A61B 17/12186; A61B 17/12113; A61L 24/02; A61L 24/06; A61B 2017/1205; A61L 2300/418; A61L 2400/06; A61L 2430/36

(57) ABSTRACT

Method of endovascular treatment of an aneurysm having a sac with a neck in fluid communication with a parent vessel. A delivery catheter is navigated through the vasculature to the aneurysm. Next, a delivery system is advanced through the delivery catheter to the aneurysm. A liquid metal embolic is injected via the delivery system into the aneurysm with the injected liquid metal embolic at least partially filling the aneurysm. Optionally, a liquid metal embolic seepage preventative element (e.g., radially self-expanding braided structure or plurality of radially self-expanding coils) may be deployed in the aneurysm prior to the injection of the liquid metal embolic prohibiting the liquid metal embolic injected in the aneurysm from entering the parent vessel.

### FIELD

The present disclosure relates to treatment of an aneurysm, and more particularly, to diverting (i.e., disrupting or impeding) blood flow from an aneurysm via use of a liquid metal embolic, optionally together with use of a liquid metal embolic seepage preventative element disposed inside of the aneurysm or in the parent vessel proximate the neck outside the aneurysm.

### BACKGROUND

An aneurysm stretches out thereby thinning a section of the wall of the artery. Cranial aneurysms may be difficult to treat due to their proximity to critical brain tissues. Conventional solutions have included endovascular treatment whereby an internal volume of the aneurysm sac is surgically removed or excluded from arterial blood pressure and flow via an endovascular intrasaccular device. Current alternatives to endovascular intrasaccular devices or other surgical approaches include endovascularly delivered treatment devices that fill the sac (i.e., dome) of the aneurysm with embolic material or block the entrance (i.e., neck) of the aneurysm. Both approaches attempt to prevent or divert blood flow into the aneurysm. By filling an aneurysm sac, the embolic material clots the blood, creating a thrombotic mass within the aneurysm. Whereas, treating the aneurysm neck, blood flow into the entrance of the aneurysm is impeded, inducing venous stasis in the aneurysm and facilitating a natural formation of a thrombotic mass within the aneurysm.

Current intravascularly delivered implantable embolization devices typically utilize multiple devices (e.g., embolic coils) to either fill the sac or treat the entrance (i.e., neck) of the aneurysm. Naturally formed thrombotic masses created by treating the entrance with embolic coils may result in improved healing compared to aneurysm masses packed with embolic coils because naturally formed thrombotic masses can reduce the likelihood of distention from arterial walls and facilitate reintegration into the original parent vessel shape along the neck plane. However, embolic coils delivered to the neck of the aneurysm can potentially have the adverse effect of impeding the flow of blood in the adjoining blood vessel, particularly if the entrance is overpacked. Conversely, if the entrance is insufficiently packed, recanalization of blood flow may persist into the aneurysm. Treating certain aneurysm morphology (e.g., wide neck, bifurcation, etc.) may require ancillary devices (e.g., stents or balloons) to support the coil mass and obtain the desired packing density. Once implanted, the coils cannot easily be retracted or repositioned. Furthermore, aneurysms treated with multiple coils over time often recanalize or compact resulting from poor coiling, lack of coverage across the aneurysm neck, blood flow, and/or relatively large aneurysm size.

Aneurysms develop into varying morphologies of different shapes and sizes. Therefore, several different size coils are needed to fill different shape and size aneurysms. It is impossible to pack an aneurysm with 100% of coils due to the interference of coils overlapping with one another. Still another concern is that the most effective occlusion rates for coils occurs when there are not gaps between the coil layers and the neck of the aneurysm. Some conventional intrasaccular devices seal the neck of the aneurysm with a braided structure, however, these devices also come in a number of varying sizes and it is difficult to achieve exactly the correct positioning of these devices at the neck of the aneurysm with no neck remnant and no protrusion of the braid structure into the parent artery or vessel.

It is therefore desirable to develop an improved simplified method for treatment of an aneurysm that address the aforementioned concerns while better conforming in size, shape and morphology of the particular aneurysm.

### SUMMARY

An aspect of the present disclosure relates to an improved method for treatment of an aneurysm using an injectable liquid metal embolic (e.g., Gallium) substantially perfectly conforming in size, shape and morphology of the particular aneurysm to be treated by injection of a liquid at least partially filling the aneurysm without causing an inflammatory response or leakage (i.e., seepage or escape) into the parent vessel.

Another aspect of the present disclosure is directed to an improved method for treatment of an aneurysm using an injectable liquid metal embolic (e.g., Gallium) at least partially filling a sac of the aneurysm. Blood present in the aneurysm is automatically self-purged via the neck into the parent vessel while being displaced by the injected liquid metal embolic.

Yet another aspect of the present disclosure is directed to an improved method for treatment of an aneurysm by at least partially filling the aneurysm with a liquid metal embolic (e.g., Gallium) and preventing seepage, leakage or escape of the injected liquid metal embolic by introducing a liquid metal embolic seepage preventative element either within the parent vessel proximate the neck outside the aneurysm or inside the aneurysm.

Still another aspect of the present disclosure relates to an improved method for treatment of an aneurysm using an injectable liquid metal embolic (e.g., Gallium) at least partially filling the aneurysm and a liquid metal embolic seepage preventive element (e.g., radially self-expanding braided structure or polymerizing agent) introduced in the parent vessel proximate the neck of the aneurysm preventing leakage, seepage or escape of the injected liquid metal embolic into the parent vessel.

While yet another aspect of the present disclosure is directed to an improved method for treatment of an aneurysm by implanting inside the aneurysm spanning the neck a liquid metal embolic seepage preventative element (e.g., radially self-expanding braided structure or coils) and thereafter injecting a liquid metal embolic (e.g., Gallium) at least partially filling the aneurysm.

Yet still another aspect of the present disclosure relates to an improved method for treatment of an aneurysm by implanting (i.e., deploying or actuating) inside the aneurysm spanning the neck a liquid metal embolic seepage preventative element (e.g., a radially self-expanding braided structure) and thereafter injecting a liquid metal embolic (e.g., Gallium) at least partially filling an interior region, section, cavity or space of the implanted liquid metal embolic seepage preventative element.

While yet still another aspect of the present disclosure relates to an improved method for treatment of an aneurysm by implanting (i.e., deploying or actuating) inside the aneurysm spanning the neck a liquid metal embolic seepage preventative element (e.g., a radially self-expanding braided structure) and thereafter injecting a liquid metal embolic (e.g., Gallium) at least partially filling inside the aneurysm exteriorly at least partially surrounding (without interiorly filling) the implanted liquid metal embolic seepage preventative element.

In yet another aspect of the present disclosure is directed to an improved method for treatment of an aneurysm by implanting (i.e., deploying or actuating) inside the aneurysm spanning the neck a liquid metal embolic seepage preventative element (e.g., a plurality of coils) and thereafter injecting a liquid metal embolic (e.g., Gallium) at least partially filling inside the aneurysm between and surrounding the coils.

### BRIEF DESCRIPTION OF THE DRAWINGS

The above and further aspects of the present disclosure are further discussed with reference to the following description in conjunction with the accompanying drawings, in which like numerals indicate like structural elements and features in various figures. The drawings are not necessarily to scale, emphasis instead being placed upon illustrating principles of the present disclosure. The figures depict one or more implementations of the devices of the present disclosure, by way of example only, not by way of limitation.

FIG. 1A is a schematic diagram of an aneurysm substantially completely filled with a liquid metal embolic which is prevented from escaping via the neck of the aneurysm via a self-expanding braided structure deployed in the parent vessel proximate outside the aneurysm proximate the neck;

FIG. 1B is a schematic diagram of an exemplary endovascular system for implanting the radially self-expanding stent in the parent vessel and injection of the liquid metal embolic inside the aneurysm, as depicted in FIG. 1A;

FIG. 2A is a schematic diagram of an example endovascular system for injection of a liquid metal embolic inside an aneurysm which is prevented from escaping via the neck of the aneurysm via a polymerizing agent deployed in the parent vessel exteriorly of the aneurysm proximate the neck;

FIG. 2B is a schematic diagram of the first stage of operation of the example endovascular system of FIG. 2A depicting injection of the liquid metal embolic inside the aneurysm and dispensing the polymerizing agent in the parent vessel, as depicted in FIG. 2A;

FIG. 2C is a schematic diagram of the second stage of operation of the example endovascular system of FIG. 2A depicting, after injection of the liquid metal embolic inside the aneurysm in FIG. 2B, dispensing the polymerizing agent in the parent vessel;

FIG. 3 is a schematic diagram of an exemplary endovascular medical system for treatment of an aneurysm in accordance with the present disclosure including a radially self-expanding braided structure advanceable through a delivery catheter via a delivery system and a syringe for injecting the liquid metal embolic dispensed via the delivery system into an interior region of the deployed radially self-expanding braided structure open at the distal end;

FIGS. 4A-4G depict the sequential stages of treatment of an aneurysm using the exemplary radially self-expanding braided structure of FIG. 3 deployed within the aneurysm and subsequent filling of the single interior region of the deployed radially self-expanding braided structure with the injected liquid metal embolic;

FIG. 5 is a schematic diagram of an exemplary endovascular medical system for treatment of an aneurysm in accordance with the present disclosure including a radially self-expanding braided structure advanceable through a microcatheter via a delivery system and a syringe for injecting the liquid metal embolic dispensed via the delivery system into an interior region of the deployed radially self-expanding braided structure closed at the distal end;

FIGS. 6A-6G depict the sequential stages of treatment of an aneurysm using the exemplary radially self-expanding braided structure of FIG. 5 deployed within the aneurysm and subsequent filling of the single interior region of the deployed radially self-expanding braided structure with the injected liquid metal embolic;

FIG. 7 is a schematic diagram still another exemplary radially self-expanding braided structure deployed inside the aneurysm; wherein the radially self-expanding braided structure has an open distal end and comprises multiple distinct radially self-expanding sections each defining a corresponding interior region; and wherein the interior region of only one of the multiple distinct radially self-expanding sections (e.g., that section disposed furthest from the neck of the aneurysm) of the braided structure is substantially completely filled with the injected liquid metal embolic;

FIG. 8 is a schematic diagram of the radially self-expanding braided structure of FIG. 7 wherein the interior region of only one of the multiple distinct radially self-expanding sections (e.g., that section disposed closest to the neck of the aneurysm) of the braided structure is substantially completely filled with the injected liquid metal embolic;

FIG. 9 is a schematic diagram of another exemplary radially self-expanding braided structure deployed inside the aneurysm; the deployed radially self-expanding braided structure including an inner braid layer and an outer braid layer defining a single interior region therebetween filled with the injected liquid metal embolic;

FIG. 10 is a schematic diagram of the same radially self-expanding braided structure of FIG. 9 wherein the liquid metal embolic is injected inside the aneurysm exteriorly of the deployed radially self-expanding braided structure;

FIG. 11 is a schematic diagram of yet another exemplary radially self-expanding braided structure deployed inside the aneurysm spanning the neck; the deployed radially self-expanding braided structure defining a single interior region filled with a liquid metal embolic;

FIG. 12 is a schematic diagram of the same radially self-expanding braided structure of FIG. 11 wherein the liquid metal embolic is injected inside the aneurysm exteriorly of the radially self-expanding braided structure;

FIG. 13 is a schematic diagram of multiple coils implanted (i.e., deployed) in the sac of the aneurysm filled with the injected liquid metal embolic surrounding and between the coils;

FIG. 14 is a flow chart of the sequential steps undertaken during the method of treatment of an aneurysm via injection of liquid metal embolic in accordance with the present disclosure;

FIG. 15 is a flow chart of the sequential steps undertaken during another method of treatment of an aneurysm via injection of liquid metal embolic in accordance with the present disclosure;

FIG. 16A is a side view of an exemplary detachment mechanism in an attached state with the delivery system secured to the radially self-expanding braided structure;

FIG. 16B is a side view of a section of the delivery system depicted in an attached state secured to the radially self-expanding braided structure in the exemplary detachment mechanism of FIG. 16A;

FIG. 16C is a radial cross-sectional view through the detachment mechanism of FIG. 16B along lines 16C-16C;

FIG. 16D is a side view of the section of the delivery system depicted in a state detached (i.e., severed or released) from the radially self-expanding braided structure in the exemplary detachment mechanism of FIG. 16A;

FIG. 17A is a side view of another exemplary detachment mechanism in an attached state with the delivery system secured to the radially self-expanding braided structure;

FIG. 17B is a side view of a section of the delivery system depicted in an attached state secured to the radially self-expanding braided structure in the exemplary detachment mechanism of FIG. 17A;

FIG. 17C is a radial cross-sectional view through the detachment mechanism of FIG. 16B along lines 17C-17C; and

FIG. 17D is a side view of the section of the delivery system depicted in a state detached (i.e., severed or released) from the radially self-expanding braided structure in the exemplary detachment mechanism of FIG. 17A.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

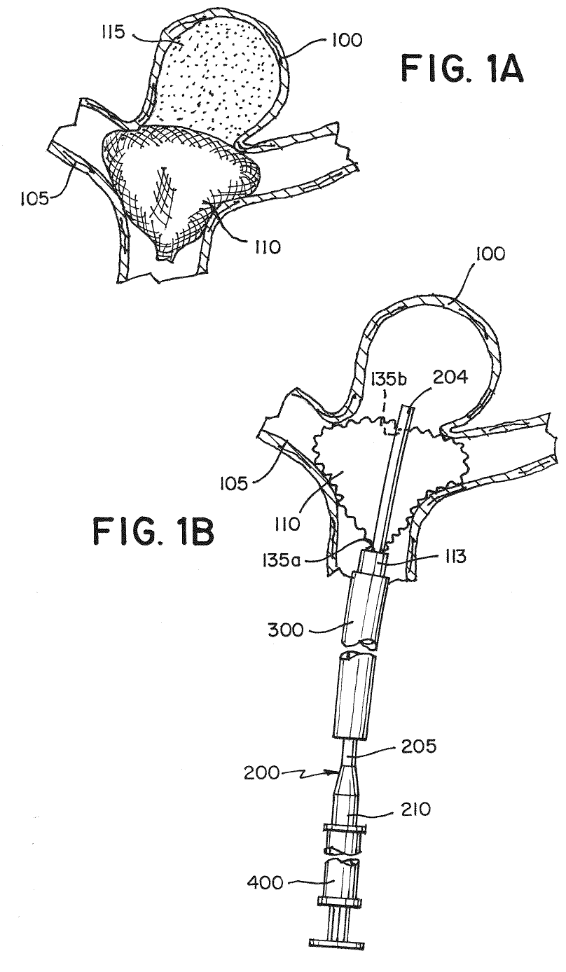

A most basic or generic aspect the present disclosure is directed to at least partial filling of the aneurysm exclusively or only with a liquid metal embolic (e.g., Gallium), i.e., without the introduction, implantation or deployment of any implantable mechanical structure inside the aneurysm. The schematic diagram of an aneurysm 100 in the example illustrated in FIG. 1A is filled at least partially, preferably completely, with the liquid metal embolic 115 (e.g., Gallium). Advantageously, the injected liquid metal embolic 115 while in a liquid state always fully, totally or completely conforms to the size, shape and morphology of the particular aneurysm in which it is injected. The liquid metal embolic 115 (e.g., Gallium) is non-toxic and thus will not trigger an inflammatory response. Regardless of whether the aneurysm 100 is filled fully or partially with the liquid metal embolic 115 (e.g., Gallium), optionally a liquid metal embolic seepage preventative element may be introduced or disposed within the parent vessel 105 outside the aneurysm 100 proximate the neck. In FIG. 1A, the liquid metal embolic seepage preventative element is a radially self-expanding braided structure 110 (e.g., radially self-expanding stent) that when implanted (i.e., deployed) in the parent vessel 105 proximate the neck of the aneurysm prevents leakage or escape of the liquid metal embolic 115 injected into the aneurysm 100. The radially self-expanding braided structure 110 is configured so that the pores defined therein (i.e., spacing between adjacent woven wires or fibers) are sized to prevent passage of the injected liquid metal embolic 115 (e.g., Gallium) into the parent vessel 105, while nevertheless allowing blood flow therethrough. Accordingly, when displaced by the injected liquid metal embolic (e.g., Gallium) blood present within the aneurysm 100 is automatically self-purged via the pores of the radially self-expanding stent implanted in the parent vessel 105 at the neck of the aneurysm 100.

FIG. 1B is an example endovascular system used during the aneurysm treatment in depicted in FIG. 1A. The endovascular system includes delivery system 200 (e.g., microcatheter 205 and luer hub 210) used to advance the radially self-expanding braided structure 110 through a delivery catheter 300 to a target site in the parent vessel 105 outside the aneurysm 100 proximate the neck (as illustrated in FIG. 1A). In an assembled or attached state the distal end of the microcatheter 205 extends completely through openings (e.g., via inlet port 135a and outlet port 135b) defined in the braided structure 110 allowing injection of the liquid metal embolic 115 via the distal end of the microcatheter 204 disposed inside the aneurysm 100 distally of the radially self-expanding braided structure 110 deployed in the parent vessel 105. In operation, the delivery catheter 300 is navigated through the vasculature to a position in the parent vessel 105 outside the aneurysm 100 proximate the neck using a guidewire that is subsequently withdrawn. While in a radially constricted state, the radially self-expanding braided structure 110 is advanced through the delivery catheter 300 using the delivery system 200 (e.g., microcatheter 205 with a luer hub 210). Upon exiting from the distal end of the delivery catheter 300, the self-expanding braided structure 110 automatically radially self-expands to a radially enlarged state (i.e., deployed or implanted state) within the parent vessel 105 outside the aneurysm 100 proximate the neck. The syringe 400 connected to the luer hub 210 is used to inject the liquid metal embolic 115 (e.g., Gallium) dispensed via the delivery system (e.g., microcatheter 205) into and at least partially filling the aneurysm 100. In all examples illustrated and described herein, both the filling and the dispensing of liquid metal embolic 115 (e.g., Gallium) via a syringe into the aneurysm occurs at substantially body temperature to ensure that the liquid metal embolic 115 is maintained in a liquid state. Displaced by the injected liquid metal embolic 115 (e.g., Gallium) blood present in the sac of the aneurysm 100 is automatically self-purged through the pores of the deployed radially self-expanding braided structure 110 into the parent vessel 105. As previously mentioned, the radially self-expanding braided structure 110 is configured so that the size of the pores (i.e., spaces defined between adjacent woven wires or fibers) defined therein allow passage therethrough of the purged blood from the sac of the aneurysm 100 while preventing leakage or seepage of the injected liquid metal embolic 115 (e.g., Gallium) from the aneurysm into the parent vessel 105. Following injection of the liquid metal embolic 115 (e.g., Gallium) the delivery system 200 (e.g., microcatheter 205) is detached from the deployed radially self-expanding stent 110 that remains implanted in the parent vessel 105. Either serially one after the other or simultaneously, the syringe 400, delivery system 200 (e.g., microcatheter 205 and luer hub 210) and delivery catheter 300 are withdrawn leaving in place the radially self-expanding braided structure 110 implanted in the parent vessel 105 outside the aneurysm 100 proximate the neck. Seepage into the parent vessel 105 of the liquid metal embolic 115 (e.g., Gallium) injected in the aneurysm 100 is prevented by the implanted radially self-expanded braided structure 110.

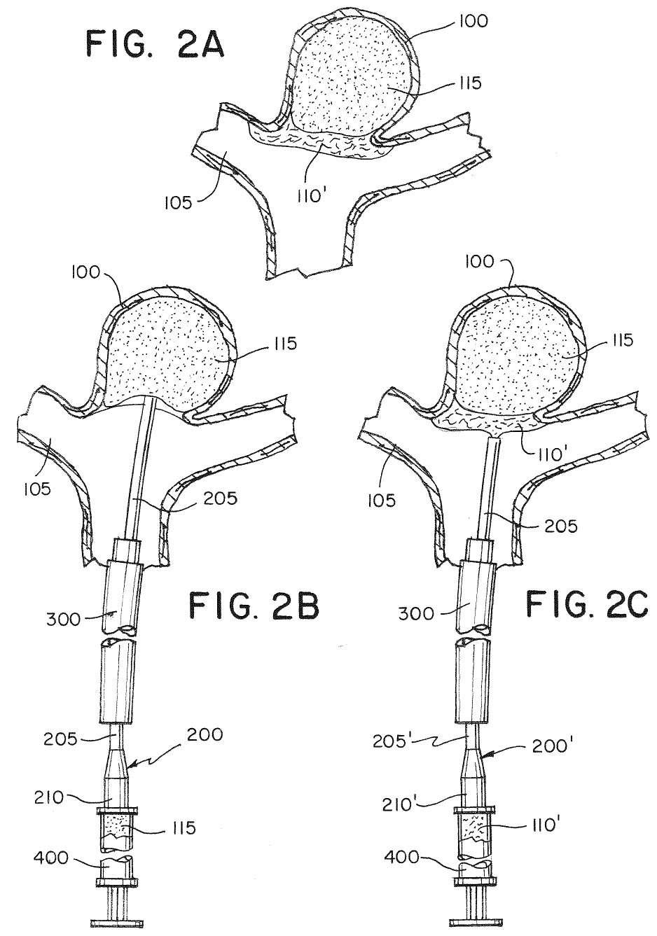

As an alternative to the implanted/deployed radially self-expanding braided structure 110, a polymerizing agent 110′ (e.g., nBCA-nButyl Cyanoacrylate) may be introduced into the parent vessel 105 outside the aneurysm 100 proximate the neck after the aneurysm is at least partially filled with the liquid metal embolic 115 (e.g., Gallium) (FIG. 2A). Polymerizing agent I10′ prevents the liquid metal embolic 115 at least partially filling the sac of the aneurysm 100 from leaking, seeping or escaping into the parent vessel 105. In contrast to the radially self-expanding braided structure 110, the polymerizing agent 110′ does not permit passage of blood flow therethrough.

The liquid metal embolic and polymerizing agent in FIG. 2A may be introduced using the example endovascular system shown in FIGS. 2B-2C. In operation, the delivery catheter 300 is navigated through the vasculature to the target site (e.g., aneurysm 100) using a guidewire that is subsequently withdrawn. The sac of the aneurysm 100 is at least partially, preferably completely, filled with the liquid metal embolic 115 (e.g., Gallium) injected via a first delivery apparatus 200 (e.g., first syringe filled with liquid metal embolic 400, first luer hub 210 and first microcatheter 205) introduced through the delivery catheter 300 (FIG. 2B). After filing (at least partially) the aneurysm 100 with the liquid metal embolic 115 (e.g., Gallium), the syringe 400 and first delivery apparatus 200 is withdrawn from the delivery catheter 300, either serially one after the other or simultaneously. Thereafter, a second delivery apparatus 200′ (e.g., second syringe filled with polymerizing agent 400′, second luer hub 210′ and second microcatheter 205') is advanced through the delivery catheter 300 to a position in the parent vessel 105 outside the aneurysm 100 proximally of the neck and the polymerizing agent 110′ is dispensed (FIG. 2C). The polymerizing agent 110′ acts like a seal preventing leakage or seepage into the parent vessel 105 of the liquid metal embolic 115 injected into the aneurysm 100. Either serially one after the other or simultaneously at the same time, the second delivery apparatus 200′ and delivery catheter 300 are withdrawn from the body. The liquid metal embolic seepage preventative element (e.g., radially self-expanding mechanical structure 110 or a polymerizing agent 110′) introduced into the parent vessel 105 outside the aneurysm 100 proximate the neck prevents leakage, seepage or escape into the parent vessel 105 of the liquid metal embolic 115 (e.g., Gallium) injected into the aneurysm 100.

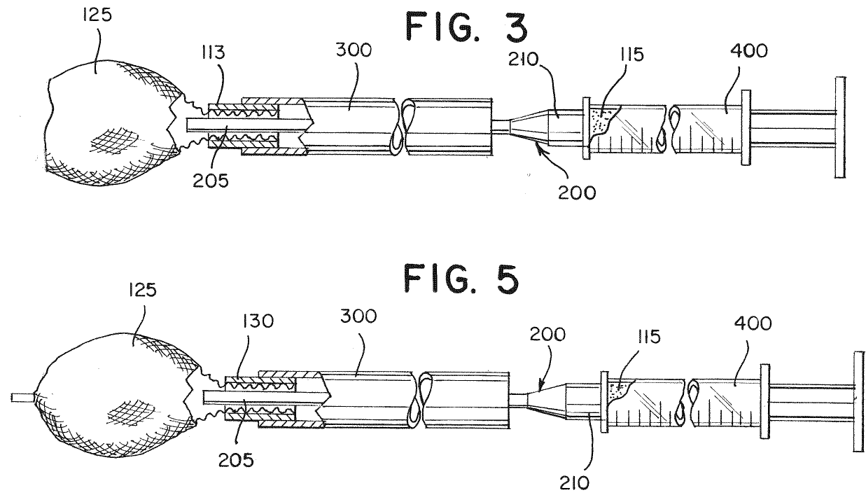

Rather than being disposed within the parent vessel 105 outside the aneurysm 100 proximate the neck, alternatively, a liquid embolic seepage preventative element (e.g., radially self-expanding braided structure or radially self-expanding coils) may be deployed inside the sac of the aneurysm 100 preferably spanning the neck. In the endovascular system depicted in FIG. 3 the liquid embolic seepage preventative element is a radially self-expanding braided structure 125 having an open distal end. The proximal end of the radially self-expanding braided structure 125 is attached via a securement element 113 (e.g., shrink sleeve, marker band, adhesive, weld, etc.) to the distal end of a delivery system 200 (e.g., distal end of microcatheter 205 with a luer hub 210). In operation, a delivery catheter 300 is navigated through the vasculature to the target aneurysm 100. While in a radially constricted state, using the delivery system 200 (e.g., microcatheter 205) the radially self-expanding braided structure 110 is advanced through the delivery catheter 300. Upon exiting from the distal end of the delivery catheter 300, the radially self-expanding braided structure 125 is deployed inside the aneurysm 100 automatically transitioning to a radially enlarged state (i.e., deployed or implanted state). Liquid metal embolic 115 (e.g., Gallium) is injected via a syringe 400 attached to the delivery system 200 (e.g., the luer hub 210) at least partially filling the interior region of the deployed radially self-expanding braided structure 125. Injection of the liquid metal embolic 115 (e.g., Gallium) in the interior region of the radially self-expanding braided structure 125 may result in further, additional or extended radial expansion relative to that of the radially self-expanding braided structure 125 prior to the injection of the liquid metal embolic 115. In addition, as the interior region of the deployed radially self-expanding braided structure 125 fills with the liquid metal embolic 115 its open distal end advantageously optimizes conformity of the deployed radially expanding braided structure 125 to the size, shape and morphology of the particular aneurysm. A further benefit provided by the open distal end of the braided structure 125 is that the size or area defined by each pore (i.e., the space defined between adjacent woven wires) is maintained substantially constant during radial expansion/contraction of the braided structure 125.

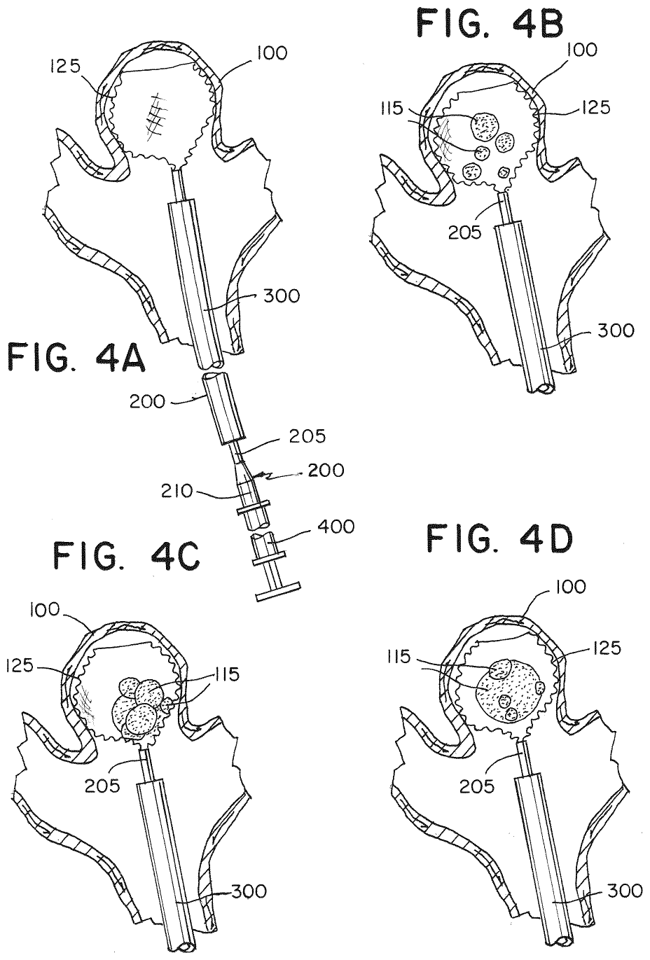

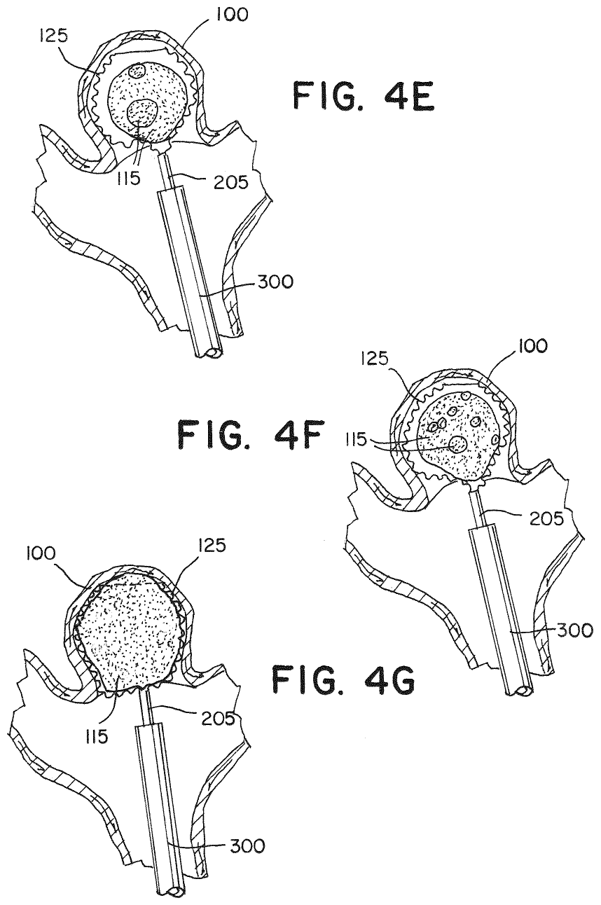

FIGS. 4A-4G depict sequential stages during treatment of the aneurysm with the radially self-expanding braided structure 125 having an open distal end using the endovascular system in FIG. 3. While in the radially constricted state the braided structure 125 attached to distal end of the delivery system 200 (e.g., microcatheter 205 with a luer hub 210) is advanced through the delivery catheter 300 to the aneurysm 100. After exiting from the distal end of the delivery catheter 300, FIG. 4A depicts braided structure 125 in a deployed or implanted state (i.e., radially enlarged state) within the aneurysm 100 while still attached to the delivery system 200 (e.g., microcatheter 205 with the luer hub 210). Syringe 400 inserted into the luer hub 210 is used to inject the liquid metal embolic 115 (e.g., Gallium) dispensed through the microcatheter 205 into an interior region of the braided structure 125 deployed in the aneurysm 100. The liquid metal embolic 115 (e.g., Gallium) preferably exhibits a sufficiently high surface tension causing separate or individual droplets within the aneurysm to adhere to one another forming larger size globules (represented in FIG. 4B by the circles 115 of varying size) filling the interior region defined by the deployed braided structure 125. As a result, the injected liquid metal embolic 115 imposes a radially outward force on the inner surface of the deployed braided structure 125 which, in turn, simultaneously pushes against the inner wall of the sac of the aneurysm 100 promoting stability while also creating a seal at the neck of the aneurysm. FIG. 4C shows the furthered adhering together of separate clumps or globules resulting from continued injection of the liquid metal embolic 115 (i.e., diminishing in number of separate globules and increasing in diameter). With still more injection of the liquid metal embolic 115 eventually the separate clumps or globules adhere together forming a single adhered clump or globule within the deployed (i.e., radially expanded) braided structure 125 (FIG. 4D). The single adhered clump or globule still continues to grow radially in diameter with the continued injection of the liquid metal embolic 115 (FIGS. 4E & 4F), until eventually substantially fully, completely or entirely filling (i.e., approximately 100%) the interior region of the deployed braided structure 125 (FIG. 4G). At this point, the delivery system 200 (e.g., microcatheter 205 and luer hub 210) is detached (e.g., mechanically, electrolytically or any other conventional detachment mechanism) from the braided structure 125 which remains in place implanted within the aneurysm 100 and filled with the liquid metal embolic 115. The syringe 400, delivery system 200 (e.g., microcatheter 204 and luer hub 210) and the delivery catheter 300 are withdrawn, either serially one after the other or simultaneously.

Despite these benefits associated with the braided structure 125 (FIG. 3) having an open distal end, it is also contemplated that the distal end of the braided structure 125 may alternatively be closed, as illustrated in the example of FIG. 5. The braided structure 125 having a closed distal end is assembled (i.e., secured)(e.g., shrink sleeve, crimped marker band, adhesive, weld, etc.) to the distal end of the delivery system 200 (e.g., microcatheter 205 and luer hub 210). The delivery catheter 300 is navigated (e.g., using a guidewire) through the vasculature to the aneurysm 100. While in a radially constricted state, using the delivery system 200 (e.g., microcatheter 205) the radially self-expanding braided structure 125 is advanced through the delivery catheter 300. Upon exiting from the distal end of the delivery catheter 300, the radially self-expanding braided structure 125 is deployed inside the sac of the aneurysm 100 automatically transitioning to a radially enlarged state. Liquid metal embolic 115 injected via syringe 400 is dispensed through the delivery system 200 (e.g., luer hub 210 and microcatheter 205) at least partially filling the interior region of the radially self-expanding braided structure 125 deployed inside the aneurysm 100. Injection of the liquid metal embolic 115 (e.g., Gallium) within the interior region of the deployed radially self-expanding braided structure 125 may result in further, additional or extended radial expansion relative to that of the deployed radially self-expanding braided structure 125 prior to the injection of the liquid metal embolic 115. Despite its distal end being closed, the deployed radially self-expanding braided structure 125 in FIG. 5 substantially conforms in size, shape and morphology to the particular aneurysm as the interior region of the radially self-expanding braided structure 125 fills with the liquid metal embolic 115.

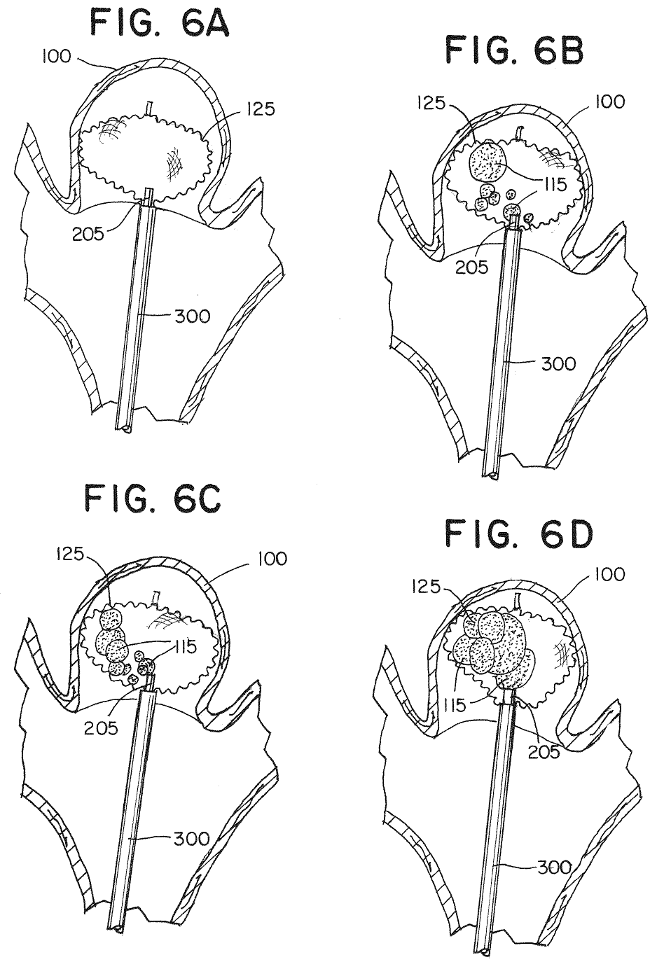

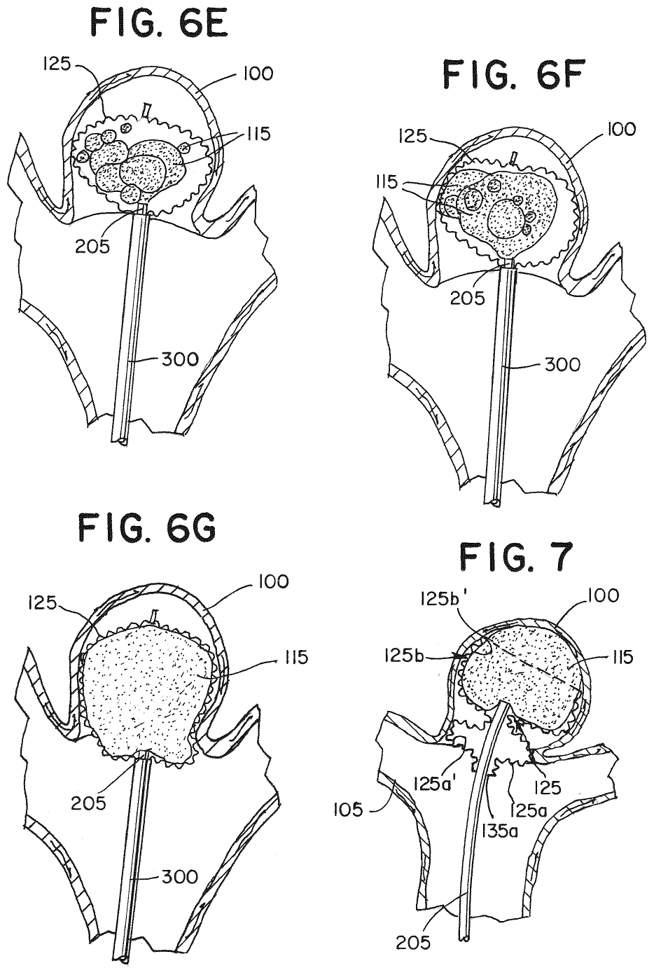

FIGS. 6A-6G depict sequential stages during treatment of the aneurysm using the radially self-expanding braided structure 125 having a closed distal end (FIG. 5). While in the radially constricted state the braided structure 125 attached to distal end of a delivery system 200 (e.g., microcatheter 205 with a luer hub 210) is advanced through the delivery catheter 300 to the target aneurysm 100. After exiting from the distal end of the delivery catheter 300, FIG. 6A depicts braided structure 125 in a deployed or implanted state (i.e., radially enlarged state) within the aneurysm 100 while still attached to the delivery system 200 (e.g., microcatheter 205 with the luer hub 210). A syringe 400 inserted into the luer hub 210 is used to inject the liquid metal embolic 115 (e.g., Gallium) dispensed through the microcatheter 205 in the interior region of the braided structure 125 deployed in the aneurysm 100. The liquid metal embolic 115 (e.g., Gallium) preferably exhibits a sufficiently high surface tension causing separate or individual droplets within the aneurysm to adhere to one another creating a larger globule (represented in FIG. 6B by the circles 115 of varying size) filling the inside of the deployed braided structure 125. As a result, the injected liquid metal embolic 115 imposes a radially outward force on the inner surface of the deployed braided structure 125 which, in turn, simultaneously pushes against the inner wall of the sac of the aneurysm providing stability while also creating a seal at the neck of the aneurysm. FIG. 6C shows the furthered adhering together of separate clumps or globules of the continued injected liquid metal embolic 115 (i.e., diminishing in number and increasing in diameter). With still more injection of the liquid metal embolic 115 eventually the separate clumps or globules adhere together forming a single adhered clump or globule within the deployed (i.e., radially expanded) braided structure 125 (FIG. 6D). The single adhered clump or globule still further grows radially in diameter with the continued injection of the liquid metal embolic 115 (FIGS. 6E & 6F), until eventually substantially fully, completely or entirely filling (i.e., approximately 100%) the interior region of the deployed braided structure 125 with a closed distal end (FIG. 6G). At this point, the delivery system 200 (e.g., microcatheter 205 and luer hub 210) is detached (e.g., mechanically, electrolytically or any other conventional detachment system) from the braided structure 125 which remains in place implanted within the aneurysm 100 and filled with the liquid metal embolic 115. The syringe 400, delivery system (e.g., microcatheter and luer hub 210) and the delivery catheter 300 are withdrawn from the body, either serially one after the other or simultaneously.

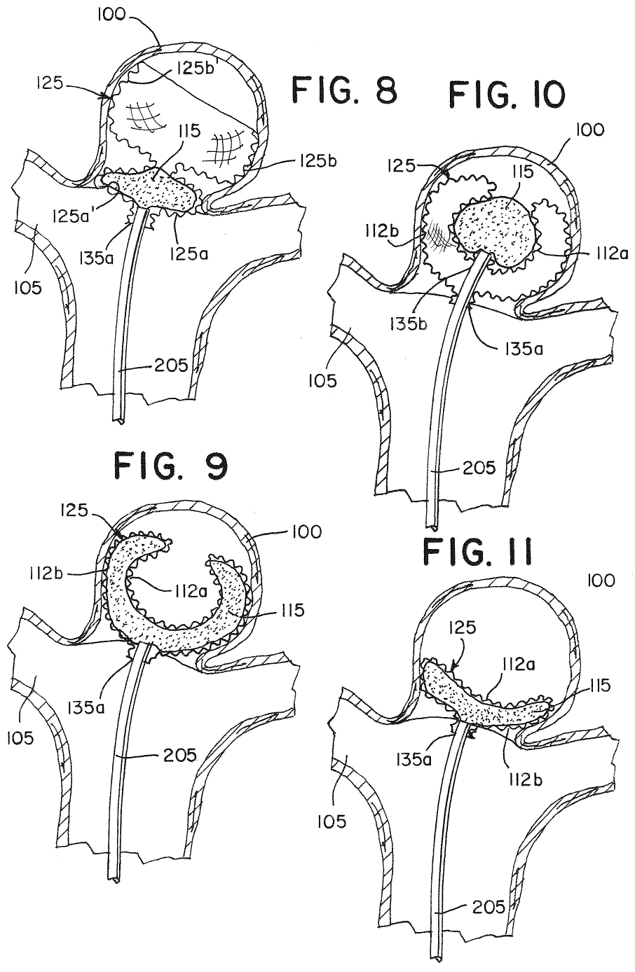

The most basic configuration of the braided structure 125 defining or forming a single interior region section, cavity or space is depicted in the examples of FIG. 3 (with an open distal end) and FIG. 5 (with a closed distal end). In those generic braided structures of FIGS. 3 & 5 the single interior region, section, cavity or space is substantially fully, completely or entirely (approximately 100%) filled with the injected liquid metal embolic 115. It should also be mentioned that partial filling (i.e., less than substantially fully, completely or entirely) of the single interior region of the deployed braided structures in FIGS. 3 & 5 is also possible. Moreover, other configurations are possible of the braided structure 125 itself (having either an open distal end or a closed distal end) forming or defining multiple interior regions, cavities or spaces. FIGS. 7 & 8 depict one such exemplary braided structure with the interior region, cavity or space forming two interior regions or sections 125a′, 125b′. In these two illustrative examples, only one of the two multiple interior regions or sections are filled with the injected liquid metal embolic 115, the other interior region or section remaining free from liquid metal embolic. Specifically, in FIG. 7 only the distal interior region or section 125b′ (farthest away from the neck of the aneurysm) of the braided structure 125 is filled with the injected liquid metal embolic 115 (as indicated by the shaded region), whereas in FIG. 8 only the proximal interior region or section 125a′ (closest to the neck of the aneurysm) of the braided structure 125 is filled with the injected liquid metal embolic 115. Of course, configuration of the braided structure 125, as desired, may define any number of multiple sections 125a, 125b, . . . , 125n at least one of which, perhaps more than one, or even all interior sections thereof 125a′, 125b′, . . . 125n′ are at least partially filled with the liquid metal embolic 115. It is still further possible that any one particular interior section of the braided structure 125 comprising multiple interior sections, may be only partially filled with the injected liquid metal embolic 115.

All the many different possible configurations of the radially self-expanding braided structure and filling of associated interior regions or sections thereof with the liquid metal embolic cannot be illustrated. Instead, certain features or concepts associated with the present disclosure are described and illustrated by way of an example configuration, but is applicable to any desired configuration, of the braided structure. For instance, FIGS. 9 & 10 depict by way of example a braided structure resembling that of a curved woven basket structure with a closed proximal end spanning the neck of the aneurysm and an opposite open distal end. In this example, the curved side of the braided structure 125 projecting substantially to the distal end of the aneurysm 100 opposite the neck maximizes contact surface area with the walls of the aneurysm thereby stabilizing the braided structure within the aneurysm. The curved woven basket structure has an inner braid layer 112a and an outer braid layer 112b defining therebetween a single interior region, section, cavity or space 115. In FIG. 9 the single interior region, section, cavity or space 115 of the braided structure 125 is filled (e.g., at least partially) with the injected liquid metal embolic 115. In operation, the delivery catheter 300 is traversed through the vasculature to the aneurysm 100. While attached to the distal end of the delivery system 200 (e.g., microcatheter 205) the braided structure 125 in the radially constricted state is advanced through the delivery catheter 300 and upon exiting therefrom is deployed inside the aneurysm 100 spanning the neck. In this example in which the liquid metal embolic 115 is injected in the interior region (i.e., at least partially filling) the deployed radially self-expanding braided structure 125, the distal end of the microcatheter 205 is secured within an inlet port 135a (e.g., proximal opening) of the braided structure 125. While the braided structure 125 is still attached to the microcatheter 205, the liquid metal embolic 115 is dispensed via a syringe 400 through the microcatheter 205 at least partially filling the interior region, section, cavity or space defined by the deployed braided structure 125. In the FIG. 9 example the deployed braided structure 125 is stabilized within the aneurysm spanning the neck creating a seal disrupting or diverting blood flow therein.

Alternatively, injection of the liquid metal embolic 115 into the aneurysm 100 may be exteriorly of (at least partially surrounding), without interiorly filling, the deployed braided structure 125. The identical braided structure 125 of FIG. 9 is presented in FIG. 10, however, the liquid metal embolic 115 is injected inside of the aneurysm 100 exteriorly of the deployed braided structure 125 (e.g., radially inward relative to the inner braid layer 112a), rather than interiorly (i.e., between the braid layers 112a, 112b). In an assembled or attached state the distal end of the microcatheter 205 passes completely (i.e., extends) through openings (e.g., via inlet port 135a and outlet port 135b) defined in the braided structure 125 allowing injection of the liquid metal embolic 115 inside the aneurysm sac exteriorly at least partially surrounding (i.e., without interiorly filling) the deployed radially self-expanding braided structure 125. In operation, the braided structure 125 while in the radially constricted state and assembled (i.e., attached) to the distal end of the microcatheter 205 is advanced through the delivery catheter 300 to the aneurysm 100. Upon exiting from the delivery catheter 300 inside the aneurysm 100 the deployed braided structure 125 radially self-expands spanning the neck of the aneurysm. The liquid metal embolic 115 injected via a syringe 400 is dispensed through the microcatheter 205 while attached to the deployed radially self-expanding braided structure 125. The aneurysm sac is at least partially filed with the injected liquid metal embolic 115 exteriorly (at least in part surrounding), without interiorly filling, the interior region, section, cavity or space of the deployed braided structure 125. In this configuration, the interior region, section, cavity or space defined between the respective inner braid layer 112a and outer braid layer 112b of the deployed braided structure being free from the injected liquid metal embolic advantageously fosters clotting of blood therein. After at least partially filling with the injected liquid metal embolic 115, the deployed braided structure 125 is detached (e.g., mechanical detachment, electrolytic detachment, etc.) from the distal end of the microcatheter 205. Either serially one after the other or simultaneously together, the syringe 400, delivery system 200 and the delivery catheter 300 are withdrawn from the body. Leakage, seepage or escape into the parent vessel 105 of the liquid metal embolic 115 injected into the aneurysm 100 is prevented by the deployed braided structure 125 spanning the neck of the aneurysm. As previously mentioned, the pores of the braided structure representing open space regions defined between adjacent wires are sized to prevent passage therethrough of the injected liquid metal embolic 115 yet allowing blood flow therethrough. Accordingly, displaced in response to injection of the liquid metal embolic 115, blood present in the aneurysm 100 is self-purged into the parent vessel 105 passing through pores defined in the deployed braided structure 125. In addition, the deployed radially self-expanding braided structure 125 free of any liquid metal embolic fosters clotting of blood between the braid layers 112a, 112b.

FIGS. 11 & 12 show still another example with the curved braided structure 125 spanning only the neck of the aneurysm. The curved braided structure includes an inner braid layer 112a and an outer braid layer 112b defining therebetween a single interior section, cavity or space. In this example in which the liquid metal embolic 115 is injected into the deployed braided structure 125, the distal end of the microcatheter 205 is secured within an inlet port 135a of the braided structure 125. Once again in FIG. 11 the liquid metal embolic 115 is injected at least partially filling the single interior section, cavity or space of the radially self-expanding braided structure 125 deployed in the aneurysm 100. The self-expanding braided structure 125 in FIG. 11 is delivered to the aneurysm using a delivery system such as that shown in FIG. 3. In operation, the delivery catheter 300 is traversed through the vasculature to the aneurysm 100 using a guidewire that is subsequently withdrawn. While in the radially constricted state the braided structure 125 while attached to the distal end of the delivery system 200 (e.g., microcatheter 205) is advanced through the delivery catheter 300 and upon exiting therefrom is deployed inside the aneurysm 100. While the braided structure 125 is still attached to the microcatheter 205, the liquid metal embolic 115 injected via the syringe 400 is dispensed through the microcatheter 205 at least partially filling the interior section, cavity or space defined by the deployed curved braided structure 125. In the FIG. 11 example the deployed curved braided structure 125 spanning the neck of the aneurysm prevents leakage, seepage or escape into the parent vessel 105 of the injected liquid metal embolic 115.

Alternatively, injection of the liquid metal embolic 115 may be inside the sac of the aneurysm exteriorly at least partially surrounding, without interiorly filling, the deployed curved braided structure 125. The identical curved braided structure 125 of FIG. 11 is presented in FIG. 12, however, the liquid metal embolic 115 is injected inside the aneurysm 100 exteriorly of the deployed braided structure. That is, the liquid metal embolic injected in the aneurysm at least partially surrounds (e.g., radially inward relative to the inner braid layer 112a), without interiorly filling the interior region defined between the inner braid layer 112a and outer braid layer 112b of the deployed curved braided structure 125. When assembled the distal end of the microcatheter 205 passes completely (i.e., extends) through the deployed curved braided structure 125 (e.g., through both inlet port 135a and outlet port 135b) allowing injection of the liquid metal embolic 115 into the sac of the aneurysm 100 exteriorly surrounding, rather than interiorly filling, the deployed curved braided structure 110. In operation, the braided structure 125 while in the radially constricted state and assembled (i.e., attached) to the delivery system 200 (e.g., microcatheter 205) is advanced through the delivery catheter 300 to the aneurysm 100. Upon exiting from the delivery catheter 300 inside the aneurysm 100 the deployed curved braided structure 125 radially expands spanning the neck of the aneurysm. The liquid metal embolic 115 injected by the syringe 400 is dispensed through the microcatheter 205 while remaining attached to the braided structure 125. The aneurysm sac 100 is at least partially filled with the injected liquid metal embolic 115 exteriorly of, without interiorly filling, the deployed braided structure 125. In this configuration, the interior section, cavity or space defined between the respective inner braid layer 112a and outer braid layer 112b of the deployed braided structure 125 being free from the injected liquid metal embolic 115 advantageously promotes clotting of blood therein. After injection of the liquid metal embolic 115, the deployed curved braided structure 125 is detached (e.g., mechanical detachment, electrolytic detachment, etc.) from the delivery system 200 (e.g., microcatheter 205). Either serially one after the other or simultaneously together, the syringe 400, delivery system 200 (e.g., microcatheter 205 with luer hub 210) and delivery catheter 300 are withdrawn from the body. Leakage, seepage or escape into the parent vessel 105 of the injected liquid metal embolic 115 is prevented by the deployed curved braided structure 125 spanning the neck of the aneurysm 100. As previously mentioned, the pores of the braided structure 125 (i.e., open space regions defined between adjacent wires or fibers) are sized to prevent passage therethrough of the injected liquid metal embolic 115 yet allowing blood flow therethrough. Accordingly, blood present in the aneurysm 100 displaced in response to the injected liquid metal embolic 115 is self-purged passing through the pores defined in the deployed braided structure 125.

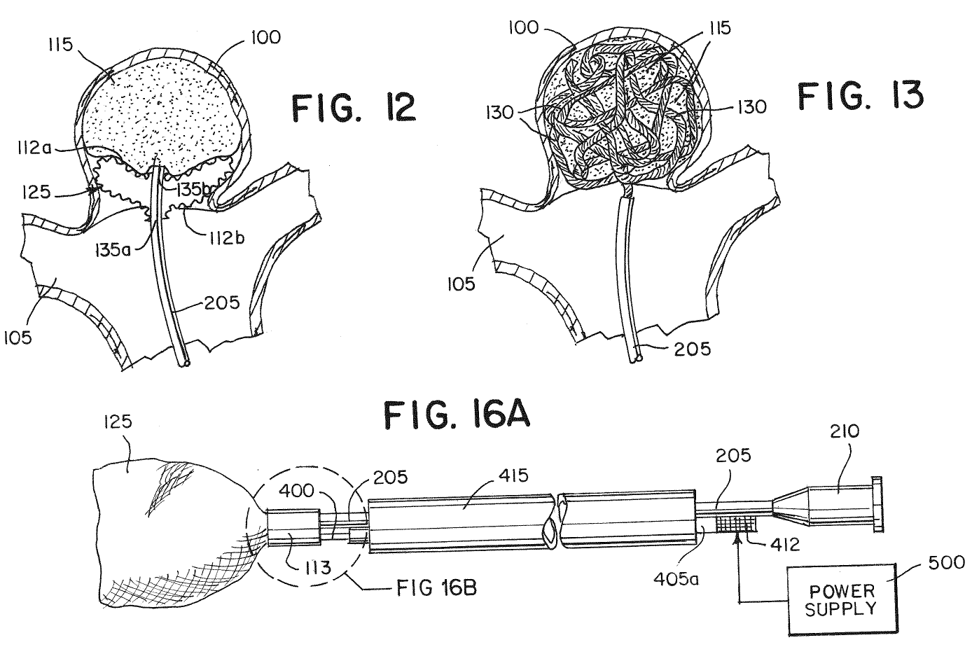

The liquid embolic seepage preventative element deployed inside the aneurysm in the examples in FIGS. 3-12 is a radially self-expanding braided structure 125. Alternatively, the aneurysm 100 may be packed with a plurality of radially self-expanding coils 130, as shown in FIG. 13. Challenges posed with use of coils 130 (e.g., achieving a desired packing density, using different size coils, inability to pack the aneurysm 100% without overlapping one another, and potential gaps between a layer of coils and the neck of the aneurysm) are addressed by at least partially filling the aneurysm with the liquid metal embolic in accordance with the present disclosure. Referring to FIG. 13, one at a time each coil 130 while in a radially constricted state is advanced through the delivery catheter 300 using the delivery system 200 (e.g., microcatheter 205). Upon exiting from the distal end of the delivery catheter 300, the coil 130 automatically radially self-expands in the sac of the aneurysm 100. The deployed coil 130 remains implanted in the aneurysm 100 following detachment and withdraw of the delivery system 200 (e.g., microcatheter 205). This delivery process is repeated for each coil 130 until the aneurysm 100 is packed with a plura1lity of coils 130. Next, the microcatheter 205 alone (without any coil 130 or other radially self-expanding braided structure secured thereto) is introduced through the delivery catheter 300 and liquid metal embolic 115 via the syringe 400 attached to the luer hub 210 is introduced into the aneurysm 100 at least partially filling the areas between and surrounding the implanted coils 130. Accordingly, those spaces created due to interference of the coils 130 overlapping one another as wells as gaps between the layers of coils and neck of the aneurysm 100 are filled with the injected liquid metal embolic 115. Furthermore, since the spaces or regions between and surrounding the coils 130 are filled with the injected liquid metal embolic 115 the aneurysm 100 may be packed with fewer coils 130 reducing overall procedure time and risks associated with overpacking. In addition, the injected liquid metal embolic 115 and deployed coils 130 together form a unitary single mass preventing future recanalization while establishing a seal at the neck of the aneurysm.

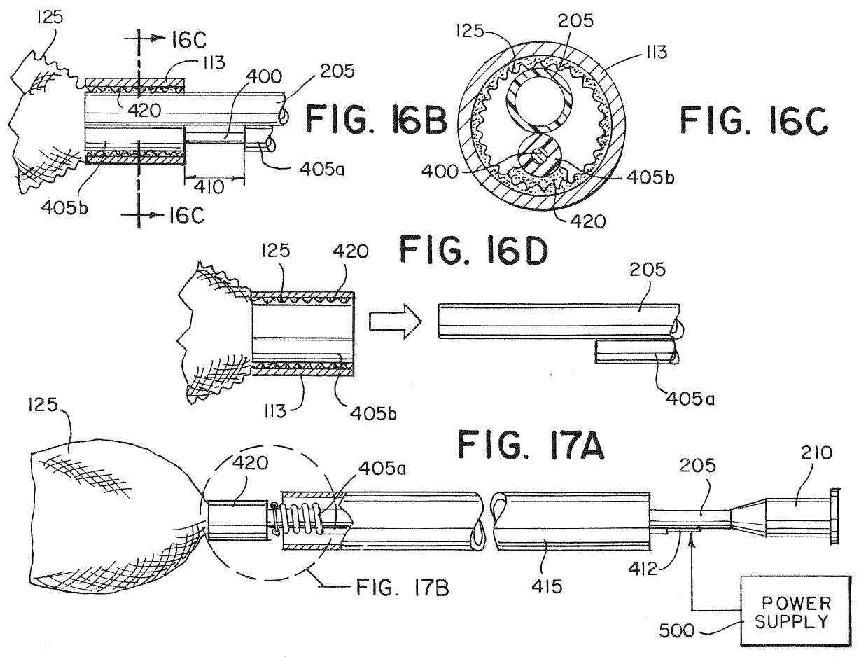

Several exemplary detachments mechanisms for delivery of the liquid metal embolic and/or radially self-expanding braided structure are illustrated and will now be described in detail, but other detachment mechanisms are contemplated and within the scope of the present disclosure. The example detachment mechanisms depict a radially self-expanding braided structure having an open distal end and a single interior cavity (similar to that in FIGS. 3 & 4A-4G), however, any desired configuration radially self-expanding braided structure may be substituted. A first example electrolytic detachment mechanism is illustrated in FIG. 16A-C (attached state) and FIG. 16D (detached or severed state). The electrolytic detachment mechanism includes a conductive metal wire 400 covered or encased by an outer jacket or layer of electrically insulated material except for a bare (i.e., exposed or electrically non-insulated) section representing a detachment zone or region 410 and a bare proximal end or section 412 electrically connected to a power supply 500. The detachment zone or region 410 is interposed between a proximal portion of the insulated electrically conductive wire 405a and a distal portion of the insulated electrically conductive wire 405b. A shrink wrap sleeve, band or other mechanical securement device 415 disposed about the delivery tube 205 and the proximal portion of the insulated electrically conductive metal wire 405a ensures that these components move simultaneously together in the axial/longitudinal direction when proximally withdrawn from the body following detachment (e.g., severing). With the distal end of the distal portion of the insulated electrically conductive wire 405b and the distal end of the delivery tube 205 disposed in a proximal end of the inner passageway of the radially self-expanding braided structure 125, the securement element 113 (e.g., marker band, sleeve, etc.) is positioned around the outer surface of the proximal end of the radially self-expanding braided structure 125 (FIG. 16C). Adhesive 420 (e.g., glue) secures together the distal portion of the insulated electrically conductive wire 405b, the radially self-expanding braided structure 125 and the securement element 113. Delivery tube 205 is intentionally not secured in any way (i.e., not adhered) to the distal portion of the insulated electrically conductive wire 405b, the radially self-expanding braided structure 125 or the securement element 113. Accordingly, in a detached state, proximal withdraw of the delivery tube 205 is allowed while the distal portion of the insulated electrically conductive wire 405b, the radially self-expanding braided structure 125 and the securement element 113 together remain implanted in position in the body. The insulated electrically conductive wire is positioned in the longitudinal/axial direction with the detachment zone or region 410 between the proximal edge of the securement element 113 and a distal edge of the shrink wrap sleeve 415. FIG. 16A-C illustrates the detachment mechanism prior to applying the electrical current with the proximal and distal portions 405a, 405b of the insulated electrically conductive wire attached via the bare electrically conductive metal wire 400 spanning the detachment zone 410. In response to applying an electrical current via the power supply 500 to the exposed section 412 of the proximal section of the insulated electrically conductive wire 405a, along the detachment zone 410 the bare, exposed or electrically non-insulated portion of the electrical wire 400 erodes separating the proximal and distal portions 405a, 405b, respectively, of the insulated electrically conductive wire from one another (FIG. 16D). The severed proximal portion of the insulated electrically conductive wire 405a and the delivery tube 205 secured together via the shrink wrap sleeve 415 are simultaneously proximally withdrawn from the body, while the severed distal portion of the insulated electrically conductive wire 405b adhered to both the deployed radially self-expanding braided structure 125 and the securement element 113 remain implanted in position (e.g., inside the aneurysm 100) in the body.

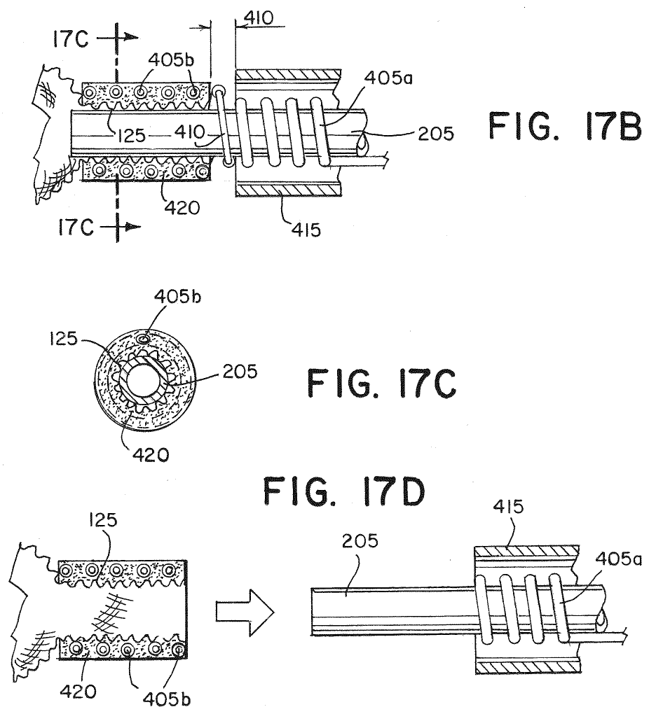

An alternative example electrolytic detachment mechanism is depicted in FIGS. 17A-C (attached state) & 17D (detached or severed state) in which the securement element 113 (e.g., marker band) may be eliminated thereby reducing the outer diameter relative to that of the example of FIG. 16A. The electrolytic detachment mechanism includes an insulated electrically conductive metal wire in which a conductive metal wire 400 is covered or encased by an outer jacket or layer of electrically insulated material except for a bare (i.e., exposed or electrically non-insulated) section representing the detachment zone or region 410 and the bare proximal end or section 412 electrically connected to the power supply 500. That portion of the exposed electrically conductive wire 400 along the detachment zone or region 410 is interposed between a proximal portion of the insulated electrically conductive wire 405a and a distal portion of the insulated electrically conductive wire 405b. With the distal end of the delivery tube 205 disposed in the inner passageway of the proximal end of the radially self-expanding braided structure 125, the insulated electrically conductive wire is coiled or wrapped about while cinching the outer surface of the proximal section of the radially self-expanding braided structure 125. In the example of FIG. 17A such coiling extends longitudinally/axially in a proximal direction about a portion of the outer surface of the delivery tube 205, but optionally may not. The extent in the longitudinal/axial direction of the wrapping of the insulated electrically conductive wire or coil may be varied with the non-insulated region 410 (i.e., detachment region) of the electrically conductive wire or coil representing a desired point, location or position of detachment. A shrink wrap sleeve, band, or other mechanical securement device 415 disposed about the delivery tube 205 and the proximal portion of the coiled insulated electrically conductive metal wire 405a ensures that these components move simultaneously together in the axial/longitudinal direction. Adhesive 420 (e.g., glue) secures together the distal portion of the coiled insulated electrically conductive wire 405b to the radially self-expanding braided structure 125 (FIG. 17A-C). Delivery tube 205 is intentionally not secured in any way (i.e., not adhered) to the distal portion of the coiled insulated electrically conductive wire 405b or the radially self-expanding braided structure 125. Accordingly, in the detached (e.g., severed) state (FIG. 17D), axial/longitudinal freely slidable movement of the delivery tube 205 together with the severed proximal portion of the coiled insulated electrically conductive wire 405a is allowed while the severed distal portion of the coiled insulated electrically conductive wire 405b and the radially self-expanding braided structure 125 remain implanted in position in the body. The detachment zone or region 410 (exposed portion of the electrically conductive metal wire 400) is arranged in the longitudinal/axial direction to align or coincide between the proximal edge of the adhesive 420 and a distal edge of the shrink wrap sleeve 415. FIG. 17A-C illustrates the detachment mechanism prior to applying the electrical current with the proximal and distal portions 405a, 405b of the coiled insulated electrically conductive wire attached via the bare electrically conductive metal wire 400 spanning the detachment zone 410. In response to applying an electrical current via the power supply 500, along the detachment zone 410 the bare, exposed or electrically non-insulated portion of the electrically conductive wire 400 erodes separating the proximal and distal portions 405a, 405b, respectively, of the coiled insulated electrically conductive wire from one another (FIG. 17D). The severed proximal portion of the coiled insulated electrically conductive wire 405a and the delivery tube 205 secured together via the shrink wrap sleeve 415 are simultaneously proximally withdrawn from the body, while the severed distal portion of the coiled insulated electrically conductive wire 405b adhered to the deployed radially self-expanding braided structure 125 remain implanted in position (e.g., inside the aneurysm 100) in the body.

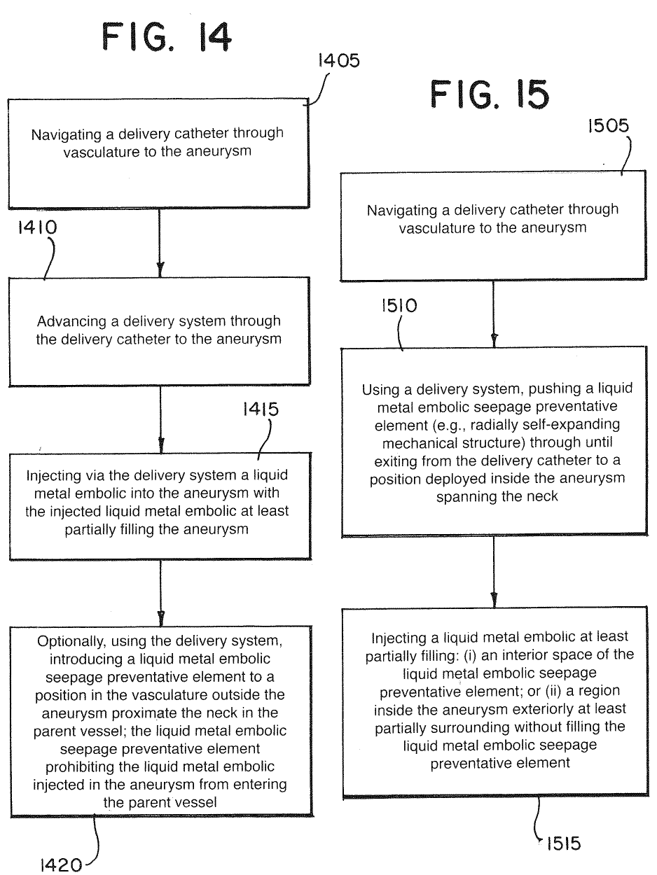

FIG. 14 is an example flow chart of the method of endovascular treatment of an aneurysm in accordance with the present disclosure. Initially, in step 1405, the delivery catheter 300 is navigated through the vasculature to the aneurysm 100 using a guidewire and subsequently withdrawn. Then, in step 1410 the delivery system 200 (e.g., microcatheter 205) is advanced through the delivery catheter 300 to the aneurysm 100. In step 1415 a liquid metal embolic 115 is injected via the syringe 400 through the delivery system 200 (e.g., microcatheter 205) into at least partially filling the aneurysm 100. Optionally, in step 1420, using the delivery system 200 (e.g., microcatheter 205) a liquid metal embolic seepage preventative element (e.g., radially self-expanding braided structure 110 or polymerizing agent 110′) is introduced to a position in the parent vessel 105 outside the aneurysm 100 proximate the neck. The liquid metal embolic seepage preventative element prohibiting the liquid metal embolic 115 injected in the aneurysm 100 from entering the parent vessel 105.

FIG. 15 is an example flow chart of another method of endovascular treatment of an aneurysm in accordance with the present disclosure. In step 1505, the delivery catheter 300 is navigated through vasculature to the aneurysm 100 to be treated using a guidewire subsequently withdrawn. Using the delivery system 200 (e.g., microcatheter 205 with luer hub 210), in step 1510 a liquid metal embolic seepage preventative element is advanced through until exiting from the delivery catheter 300 to a position deployed inside the aneurysm 100 spanning the neck. The liquid metal embolic seepage preventative element is preferably a radially self-expanding mechanical structure (e.g., radially self-expanding braided structure 125 or coils 130). Lastly, in step 1515, the liquid metal embolic 1515 dispensed via the delivery system 200 (e.g., luer hub 210 and microcatheter 205) at least partially fills: (i) an interior region, section, cavity or space of the deployed liquid metal embolic seepage preventative element 125 (e.g., radially self-expanding braided structure); or (ii) inside the sac of aneurysm exteriorly of, without interiorly filling, the deployed liquid metal embolic seepage preventative element 125 (e.g., radially self-expanding braided structure 125). Thereafter, the delivery system 200 (e.g., microcatheter 205) is detached (e.g., mechanically, electrolytically, etc.) from the deployed radially self-expanding braided structure. Either serially one after the other or simultaneously, the syringe 400, delivery system 200 (e.g., luer hub 210 and microcatheter 205), and delivery catheter 300 are withdrawn from the body.

Aspects of the present disclosure are also provided by the following numbered Clauses:
Clause 1: A method of endovascular treatment of an aneurysm (100) having a sac with a neck in fluid communication with a parent vessel (105), the method comprising the steps of: navigating a delivery catheter (300) through the vasculature to the aneurysm (100); advancing a delivery system (200) through the delivery catheter (300) to the aneurysm (100); and injecting via the delivery system (200) a liquid metal embolic (115) into the aneurysm (100) with the injected liquid metal embolic (115) at least partially filling the aneurysm (100).
Clause 2: The method of Clause 1, wherein the liquid metal embolic (115) is Gallium.
Clause 3: The method of any of Clauses 1 through 2, wherein the injected liquid metal embolic (115) at least partially fills the aneurysm (100).
Clause 4: The method of any of Clauses 1 through 3, wherein the delivery system (200) delivers only the injected liquid metal embolic (115) whereby only the injected liquid metal embolic (115) is present inside the aneurysm (100); and in response to the injection of the liquid metal embolic (115), blood present in the aneurysm (100) is automatically self-purged via the neck into the parent vessel (105).
Clause 5: The method of Clause 1, further comprising using the delivery system (200), introducing a liquid metal embolic seepage preventative element (110, 110′) to a position in the vasculature outside the aneurysm (100) proximate the neck in the parent vessel (105); the liquid metal embolic seepage preventative element (110, 110') prohibiting the liquid metal embolic (115) injected in the aneurysm (100) from entering the parent vessel (105).
Clause 6: The method of Clause 5, wherein the liquid metal embolic seepage preventive element is a radially self-expanding braided structure (110) or polymerizing agent (110').
Clause 7: The method of Clause 6, wherein the introducing step comprises the steps of: pushing through the delivery catheter (300) using the delivery system (200) the radially self-expanding braided structure (110) while in a radially constricted state; and upon exiting from the delivery catheter (300), deploying in the parent vessel (105) outside the aneurysm (100) proximate the neck the radially self-expanding braided structure (110) automatically transitioning to a radially expanded state; wherein the deployed radially self-expanding braided structure (110) is configured to have pores sized to prevent seepage therethrough of the injected liquid metal embolic (115) into the parent vessel (105) while allowing blood to escape from inside the aneurysm (100) into the parent vessel (105).
Clause 8: The method of Clause 6, wherein the polymerizing agent (110′) is n-Butyl Cyanoacrylate.
Clause 9: The method of Clause 1, wherein prior to the injecting step further comprising: using the delivery system (200), pushing a liquid metal embolic seepage preventative element (125) through the microcatheter (300) to a position deployed inside the aneurysm (100) spanning the neck of the aneurysm (100); the liquid metal embolic seepage preventative element (125) prohibiting the liquid metal embolic (115) injected in the aneurysm (100) from entering the parent vessel (105); wherein the liquid metal embolic seepage preventative element is a radially self-expanding braided structure (125) or a plurality of radially self-expanding coils (130).
Clause 10: The method of Clause 9, wherein the deployed radially self-expanding braided structure (125) is configured to have: (i) at least one radially self-expanded section (125a, 125b, . . . , 125n) defining an associated interior region (125a′, 125b′, . . . , 125n′); and (ii) pores sized to prevent seepage therethrough of the injected liquid metal embolic (115) while allowing blood to escape from inside the aneurysm (100) into the parent vessel (105).
Clause 11: The method of Clause 10, wherein the radially self-expanding braided structure (125) has at least two radially self-expandable sections (125a, 125b, . . . , 125n) including a radially self-expandable proximal section (125a) and at last one supplemental radially self-expandable section (125b) disposed distally thereof the radially self-expandable proximal section (125a); each of the at least two radially self-expandable sections (125a, 125b) defining the associated interior region (125a′, 125b′).
Clause 12: The method of ay of Clauses 10 through 11, wherein the injected liquid metal embolic (115) only fills at least one of the interior regions among the associated interior regions (125a′, 125b′, . . . , 125n′) of the at least one radially self-expanded section (125a, 125b, . . . , 125n) of the deployed self-expanding braided structure (125).
T Clause 13: he method of Clause 10, wherein the injected liquid metal embolic (115) at least partially fills the aneurysm (100) only exteriorly of the deployed radially self-expanding braided structure (125); the injected liquid metal embolic (115) pushing in a proximal direction against the deployed self-expanding braided structure (125) spanning the neck of the aneurysm (100) creating a seal from inside the aneurysm (100).
Clause 14: The method of Clause 10, wherein the injected liquid metal embolic (115) at least partially fills the aneurysm (100) exteriorly of the deployed self-expanding braided structure (125); the injected liquid metal embolic (115) stabilizing from inside the aneurysm (100) the deployed self-expanding braided structure (125) spanning the neck of the aneurysm (100) preventing migration of the deployed self-expanding braided structure (125) into the parent vessel (105).
Clause 15: The method of any of Clauses 10 through 14, wherein neither the injected liquid metal embolic (115) nor the self-expanding braided structure (125) deployed inside the aneurysm (100) either enters or disrupts blood flow through the parent vessel (105).
Clause 16: The method of any of Clauses 10 through 15, wherein in response to injecting the liquid metal embolic (115) into the aneurysm (100), further comprising self-purging via the pores of the radially self-expanding braided structure (125) blood from inside the aneurysm (100) into the parent vessel (105).
Clause 17: The method of Clause 9, wherein the pushing step comprises the step of: packing the aneurysm (100) with the plurality of radially self-expanding coils (130) delivered sequentially through the delivery catheter (300) using the delivery system (200); each of the plurality of radially self-expanding coils transitioning to a radially self-expanded state inside the aneurysm (100) upon exiting from the delivery catheter (300); wherein the injected liquid metal embolic (115) fills the aneurysm (100) surrounding and in between the deployed plurality of radially self-expanding coils (130); and blood present inside the aneurysm (100) is automatically self-purged via the neck of the aneurysm (100) into the parent vessel (105) in response to the injection of the liquid metal embolic (115) into the aneurysm (100).
Clause 18: The method of Clause 17, wherein neither the liquid metal embolic (115) injected in the aneurysm (100) nor the plurality of radially self-expanding coils (130) deployed inside the aneurysm (100) either enters or disrupts blood flow through the parent vessel (105).
Clause 19: The method of Clause 9, further comprising the steps of: detaching via an electrolytic detachment mechanism the delivery system (200) from the deployed liquid metal embolic seepage preventative element (125); the electrolytic detachment mechanism including an insulated electrically conductive wire comprising a distal portion insulated electrically conductive wire (405b), a proximal portion insulated electrically conductive wire (405a) and a non-insulated portion electrically conductive wire interposed between the distal portion of the insulated electrically conductive wire (405b) and the proximal portion of the insulated electrically conductive wire (405a) defining a detachment zone (410) along which the non-insulated portion electrically conductive wire erodes in response to application of an electrical current; and withdrawing simultaneously the delivery system (200) together with the detached proximal portion of the insulated electrically conductive wire (405a) from the delivery catheter (300), while the deployed liquid metal embolic seepage preventative element (125) and the detached distal portion of the insulated electrically conductive wire (405b) remain in place.
Clause 20: The method of Clause 19, wherein the distal portion of the insulated electrically conductive wire (405b) is coiled about cinching an outer surface of the proximal end of the liquid metal embolic seepage preventative element (125).

The descriptions contained herein are examples and not intended in any way to limit the scope of the present disclosure. As described herein, the present disclosure contemplates many variations and modifications of the method for treatment of an aneurysm at least partially filled with a liquid metal embolic (e.g., Gallium). Optionally, a liquid metal embolic seepage preventative device may be deployed outside the aneurysm in the parent vessel or inside the aneurysm to prevent leakage, seepage or escape into the parent vessel of the injected liquid metal embolic inside the aneurysm. Modifications and variations apparent to those having skilled in the pertinent art according to the teachings of this disclosure are intended to be within the scope of the claims which follow.

What is claimed is:

1. A method of endovascular treatment of an aneurysm having a sac with a neck in fluid communication with a parent vessel, the method comprising the steps of:
navigating a delivery catheter through the vasculature to the aneurysm;
advancing a delivery system through the delivery catheter to the aneurysm; and
injecting via the delivery system a liquid metal embolic into the aneurysm with the injected liquid metal embolic at least partially filling the aneurysm.

2. The method in accordance with claim 1, wherein the liquid metal embolic is Gallium.

3. The method in accordance with claim 1, wherein the injected liquid metal embolic at least partially fills the aneurysm.

4. The method in accordance with claim 3, wherein the delivery system delivers only the injected liquid metal embolic whereby only the injected liquid metal embolic is present inside the aneurysm; and in response to the injection of the liquid metal embolic, blood present in the aneurysm is automatically self-purged via the neck into the parent vessel.

5. The method in accordance with claim 1, further comprising:
using the delivery system, introducing a liquid metal embolic seepage preventative element to a position in the vasculature outside the aneurysm proximate the neck in the parent vessel; the liquid metal embolic seepage preventative element prohibiting the liquid metal embolic injected in the aneurysm from entering the parent vessel.

6. The method in accordance with claim 5, wherein the liquid metal embolic seepage preventive element is a radially self-expanding braided structure or polymerizing agent.

7. The method in accordance with claim 6, wherein the introducing step comprises the steps of:
pushing through the delivery catheter using the delivery system the radially self-expanding braided structure while in a radially constricted state; and
upon exiting from the delivery catheter, deploying in the parent vessel outside the aneurysm proximate the neck the radially self-expanding braided structure automatically transitioning to a radially expanded state; wherein the deployed radially self-expanding braided structure is configured to have pores sized to prevent seepage therethrough of the injected liquid metal embolic into the parent vessel while allowing blood to escape from inside the aneurysm into the parent vessel.

8. The method in accordance with claim 6, wherein the polymerizing agent is n-Butyl Cyanoacrylate.

9. The method in accordance with claim 1, wherein prior to the injecting step further comprising:
using the delivery system, pushing a liquid metal embolic seepage preventative element through the microcatheter to a position deployed inside the aneurysm spanning the neck of the aneurysm; the liquid metal embolic seepage preventative element prohibiting the liquid metal embolic injected in the aneurysm from entering the parent vessel; wherein the liquid metal embolic seepage preventative element is a radially self-expanding braided structure or a plurality of radially self-expanding coils.

10. The method in accordance with claim 9, wherein the deployed radially self-expanding braided structure is configured to have: (i) at least one radially self-expanded section defining an associated interior region; and (ii) pores sized to prevent seepage therethrough of the injected liquid metal embolic while allowing blood to escape from inside the aneurysm into the parent vessel.

11. The method in accordance with claim 10, wherein the radially self-expanding braided structure has at least two radially self-expandable sections including a radially self-expandable proximal section and at last one supplemental radially self-expandable section disposed distally thereof the radially self-expandable proximal section; each of the at least two radially self-expandable sections defining the associated interior region.

12. The method in accordance with claim 10, wherein the injected liquid metal embolic only fills at least one of the interior regions among the associated interior regions of the at least one radially self-expanded section of the deployed self-expanding braided structure.

13. The method in accordance with claim 10, wherein the injected liquid metal embolic at least partially fills the aneurysm only exteriorly of the deployed radially self-expanding braided structure; the injected liquid metal embolic pushing in a proximal direction against the deployed self-expanding braided structure spanning the neck of the aneurysm creating a seal from inside the aneurysm.

14. The method in accordance with claim 10, wherein the injected liquid metal embolic at least partially fills the aneurysm exteriorly of the deployed self-expanding braided structure; the injected liquid metal embolic stabilizing from inside the aneurysm the deployed self-expanding braided structure spanning the neck of the aneurysm preventing migration of the deployed self-expanding braided structure into the parent vessel.

15. The method in accordance with claim 10, wherein neither the injected liquid metal embolic nor the self-expanding braided structure deployed inside the aneurysm either enters or disrupts blood flow through the parent vessel.

16. The method in accordance with claim 10, wherein in response to injecting the liquid metal embolic into the aneurysm, further comprising self-purging via the pores of the radially self-expanding braided structure blood from inside the aneurysm into the parent vessel.

17. The method in accordance with claim 9, wherein the pushing step comprises the step of:
packing the aneurysm with the plurality of radially self-expanding coils delivered sequentially through the delivery catheter using the delivery system; each of the plurality of radially self-expanding coils transitioning to a radially self-expanded state inside the aneurysm upon exiting from the delivery catheter;
wherein the injected liquid metal embolic fills the aneurysm surrounding and in between the deployed plurality of radially self-expanding coils; and blood present inside the aneurysm is automatically self-purged via the neck of the aneurysm into the parent vessel in response to the injection of the liquid metal embolic into the aneurysm.

18. The method in accordance with claim 17, wherein neither the liquid metal embolic injected in the aneurysm nor the plurality of radially self-expanding coils deployed inside the aneurysm either enters or disrupts blood flow through the parent vessel.

19. The method in accordance with claim 9, further comprising the steps of:
detaching via an electrolytic detachment mechanism the delivery system from the deployed liquid metal embolic seepage preventative element; the electrolytic detachment mechanism including an insulated electrically conductive wire comprising a distal portion insulated electrically conductive wire, a proximal portion insulated electrically conductive wire and a non-insulated portion electrically conductive wire interposed between the distal portion of the insulated electrically conductive wire and the proximal portion of the insulated electrically conductive wire defining a detachment zone along which the non-insulated portion electrically conductive wire erodes in response to application of an electrical current; and
withdrawing simultaneously the delivery system together with the detached proximal portion of the insulated electrically conductive wire from the delivery catheter, while the deployed liquid metal embolic seepage preventative element and the detached distal portion of the insulated electrically conductive wire remain in place.

20. The method in accordance with claim 19, wherein the distal portion of the insulated electrically conductive wire is coiled about cinching an outer surface of the proximal end of the liquid metal embolic seepage preventative element.
